(19) United States
(12) Patent Application Publication
(10) Pub. No.: US 2008/0139792 A1
(43) Pub. Date: Jun. 12, 2008

(54) High Protein Concentration Formulations Containing Mannitol

(75) Inventors: David C. Sek, North Andover, MA (US); Kin Ho, Andover, MA (US)

Correspondence Address: CHOATE, HALL & STEWART LLP/WYETH, PATENT GROUP, TWO INTERNATIONAL PLACE, BOSTON, MA, 02110, US

(73) Assignee: WYETH, Madison, NJ (US)

(21) Appl. No.: 11/950,986

(22) Filed: Dec. 5, 2007

Related U.S. Application Data
(60) Provisional application No. 60/873,526, filed on Dec. 6, 2006.

(51) Int. Cl. C07K 16/00; C07K 2/00

(52) U.S. Cl. 530/391.1; 530/350; 530/386

(57) ABSTRACT

The present invention provides a method for inhibiting mannitol-induced aggregation of a protein in a liquid formulation by increasing the protein concentration to an amount greater than 50 mg/ml. The present invention also provides methods for storing and preparing a liquid formulation containing mannitol and a protein concentration greater than 50 mg/ml.

### REFERENCE TO RELATED APPLICATIONS

This application claims the benefit of U.S. Provisional Patent Application No. 60/873,526, filed on Dec. 6, 2006, which is hereby incorporated by reference in its entireties.

### FIELD OF THE INVENTION

The present invention relates to methods for storing and preparing protein formulations containing mannitol.

### BACKGROUND OF THE INVENTION

Mannitol has been generally used in protein formulations for maintaining stability and isotonicity of the formulation. In the past, liquid nitrogen has been used to quickly freeze protein formulations for storage. However, nearly all approaches to large-scale uncontrolled freezing of liquid formulations suffer from negative effects of uncontrolled solidification and melting. Inadequate control of phase change has been shown to result in product losses due to aggregation, precipitation, oxidation and denaturation. Recent technologies have been introduced to control the freeze and thaw process of protein formulations. These technologies typically freeze and thaw at a much slower rate. As a result, in mannitol-containing protein formulations, the slow freeze-thaw process allows crystallization of mannitol which, in turn, induces protein aggregation. In order to avoid mannitol-induced protein aggregation during slow freeze-thaw processes, existing methods require removing mannitol from protein formulations and adding it back during post-thaw operation.

### SUMMARY OF THE INVENTION

The present invention provides an improved method for storing and preparing protein formulations containing mannitol. Specifically, the method of the present invention permits frozen storage of protein formulations containing mannitol without first removing mannitol. Therefore, the present invention reduces costs and processing steps and time for storing and preparing protein formulations containing mannitol.

In one aspect, the present invention provides a method for storing a liquid formulation including gradually cooling the liquid formulation to a temperature lower than about −10° C. The liquid formulation contains mannitol and a protein, the protein being in a concentration greater than 50 mg/ml such that the greater concentration suppresses protein aggregation during cooling.

In one embodiment, the method of the present invention includes gradually cooling the liquid formulation to a temperature lower than about −20° C. In another embodiment, the method of the present invention includes gradually cooling the liquid formulation to a temperature at approximately −40° C. or lower. In yet another embodiment, the method of the present invention includes gradually cooling the liquid formulation to a temperature at approximately −50° C. or lower.

In some embodiments, the present invention can be used for storing the liquid formulations containing mannitol in an amount ranging approximately 0-15%. In particular, the liquid formulation may contain mannitol in an amount of approximately 1%, 2%, 3%, 4%, 5%, 6%, 7%, 8%, 9%, 10%, 11%, 12%, 13%, 14% or 15%. Percentages are weight/weight when referring to solids and weight/volume when referring to liquids.

In some embodiments, the method of the present invention includes gradually cooling the liquid formulation at a rate of approximately 0.6, 0.5, 0.4, 0.3, 0.2, or 0.1° C./minute.

In some embodiments, the liquid formulation contains a protein in a concentration greater than about 75 mg/ml, 100 mg/ml, 125 mg/ml, 150 mg/ml, or 200 mg/ml. Preferably, the liquid formulation contains a protein in a concentration between 50 mg/ml and 200 mg/ml.

In some embodiments, the liquid formulation contains a protein that is an antibody. In particular, the antibody is a monoclonal antibody. In other embodiments, the liquid formulation contains a protein that is a pharmaceutical drug substance.

In some embodiments, the method for storing a liquid formulation of the present invention is a process intermediate.

In another aspect, the present invention provides a method for preparing a liquid formulation including gradually warming the liquid formulation from a frozen state to a temperature higher than about 0° C. The liquid formulation contains mannitol and a protein in a concentration greater than 50 mg/ml such that the greater concentration suppresses protein aggregation during warming.

In some embodiments, the method for preparing a liquid formulation includes gradually warming the liquid formulation from a frozen state to a temperature at approximately 10° C., 20° C., 25° C., 30° C. or higher.

In some embodiments, the present invention can be used for preparing the liquid formulations containing mannitol in an amount ranging approximately 0-15%. In particular, the liquid formulation contains mannitol in an amount of approximately 1%, 2%, 3%, 4%, 5%, 6%, 7%, 8%, 9%, 10%, 11%, 12%, 13%, 14% or 15%. Percentages are weight/weight when referring to solids and weight/volume when referring to liquids.

In some embodiments, the method for preparing a liquid formulation includes gradually warming the liquid formulation at a rate of approximately 0.6, 0.5, 0.4, 0.3, 0.2, or 0.1° C./minute.

In some embodiments, the liquid formulation contains a protein in a concentration greater than about 75 mg/ml, 100 mg/ml, 125 mg/ml, 150 mg/ml, or 200 mg/ml. Preferably, the liquid formulation contains a protein in a concentration between 50 mg/ml and 200 mg/ml.

In some embodiments, the liquid formulation contains a protein that is an antibody. In particular, the antibody is a monoclonal antibody. In other embodiments, the liquid formulation contains a protein that is a pharmaceutical drug substance.

In some embodiments, the method for preparing a liquid formulation of the present invention is a process intermediate.

The liquid formulation of the present invention is normally an aqueous formulation.

The present invention further provides a composition containing a biologically effective amount of the protein in the liquid formulation prepared by the method of the invention as described in various embodiments above.

In yet another aspect, the present invention provides a method for inhibiting mannitol-induced aggregation of a protein in a liquid formulation by increasing the protein concentration to an amount greater than 50 mg/ml. In some embodiments, the method of the present invention inhibits mannitol-induced aggregation of a protein in a liquid formulation by increasing the protein concentration to an amount greater than about 75 mg/ml, 100 mg/ml, 125 mg/ml, 150 mg/ml, or 200 mg/ml. Typical protein concentration is between 50 mg/ml and 200 mg/ml.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

The drawings are for illustration purposes only, not for limitation.

FIG. 1 illustrates a sample product temperature trace at each exemplary process scale with a CryoPilot (CP) system.

FIG. 2 shows X-ray diffraction (XRD) patterns of frozen antibody solutions when cooled to −40° C. then warmed to 20° C. both at 0.5° C./minute.

FIG. 3 shows a sample thermogram of modulated differential scanning calorimetry (mDSC) when cooling a monoclonal antibody at concentration of 30 mg/ml down to −42° C.

FIG. 4 depicts total enthalpy plotted against protein concentration during a slow freeze and thaw process.

FIG. 5 depicts size-exclusive chromatography HPLC (SEC-HPLC) chromatograms of representative antibodies.

FIG. 6 depicts change in the percentage of high molecular weight (HMW) species plotted against the protein concentration.

FIG. 7 depicts that same freeze/thaw profile with same mixing speed resulted in 2 sets of traces based on production loads.

FIG. 8 depicts that the rates for faster freeze and thaw of the lab system were faster than the rates at the minimum production load.

FIG. 9 depicts that the rates for slow freeze and thaw of the lab system were slower than the rates at maximum production scale.

FIG. 10 depicts a typical supercooling phenomenon observed during lab scale cycle development for slow freeze and thaw.

FIG. 11 depicts that the revised profile was performed on 5 buffer trials, followed by 5 MabM trials with or without mannitol (15 total) and no supercooling was observed in any of the 10 thermocouple traces (0% occurrence).

FIG. 12 depicts that the product temperature traces of Mab and MabM overlayed and no supercooling was observed for any of the 10 thermocouple traces up to 5 Mab runs with or without mannitol.

FIG. 13 illustrates that the percentage of HMW species increased more significantly after multiple freeze and thaw cycles at slow rates as compared to fast rates.

FIG. 14 illustrates that no increase in HMW species was observed with Mab formulation at concentration of 100 mg/mL containing mannitol.

### DETAILED DESCRIPTION OF THE INVENTION

The present invention provides an improved method for storing and preparing protein formulations containing mannitol. Specifically, the present invention provides a method for suppressing or eliminating mannitol-induced protein aggregation in a liquid formulation during slow freeze and/or thaw process by increasing protein concentration.

Various aspects of the invention are described in further detail in the following subsections. The use of subsections is not meant to limit the invention. Each subsection may apply to any aspect of the invention.

### Protein Formulations Containing Mannitol

Proteins are relatively unstable in the aqueous state and undergo chemical and physical degradation resulting in a loss of biological activity during processing and storage. Freeze-thaw and lyophilisation are well-established methods for preserving proteins for storage. In order to preserve protein conformation, activity and stability, the protein formulations usually contain agents facilitating this, so-called lyoprotectants and cryoprotectants. Cryoprotectants are agents which provide stability to the protein from freezing-induced stresses; however, the term also includes agents that provide stability, e.g., to bulk drug formulations during storage from non-freezing-induced stresses. Lyoprotectants are agents that provide stability to the protein during water removal from the system during the drying process, presumably by maintaining the proper conformation of the protein through hydrogen bonding. Cryoprotectants can also have lyoprotectant effects. Examples of frequently used bulking agents include mannitol, glycine, sucrose, lactose, etc. The agents also contribute to the tonicity of the formulations.

As used herein, “proteins” include any recombinant or purified polypeptides including, but not limited to, antibodies, e.g., monoclonal antibodies, single chain antibodies, and other antibody variants; various growth hormones; and any pharmaceutical drug substances. Proteins referred to in this application include any naturally-occurring, modified or synthesized polypeptides.

As used herein, “a protein formulation,” “a liquid formulation,” or grammatical equivalents include any liquid polypeptide-containing compositions. Typically, a liquid formulation of the invention is an aqueous formulation. The liquid polypeptide-containing compositions may further contain “buffering agent” including those agents which maintain the solution pH in an acceptable range and may include bulking agents described above and may also include histidine, phosphate, citrate, tris, diethanolamine, and the like. If the liquid polypeptide-containing compositions are pharmaceutical compositions, the liquid formulation may further contain “excipients.” The term “excipients” includes pharmaceutical acceptable carriers as well as lyoprotectants and cryoprotectants that provide proper conformation of the protein during storage so that substantial retention of biological activity and protein stability is maintained.

### Mannitol Induces Protein Aggregation During Slow Freeze and Thaw

As discussed above, freeze and thaw is a well establish method for long-term storage or as an intermediate step. However, nearly all approaches to large-scale freezing of liquid formulations suffer from negative effects of uncontrolled solidification and melting. Approaches such as freezing in bags and bottles have been repeatedly shown to result in cryoconcentration and non-uniform temperature profiles within containers. Inadequate control of phase change has been shown to result in product losses due to aggregation, precipitation, oxidation and denaturation. By contrast, controlled freeze and thaw (also referred to as slow freeze and thaw) avoids product denaturation typical of uncontrolled methods and eliminates expensive and time-consuming cleaning. In addition, overall processes benefit from a well-controlled and predictable operation.

Controlled freezing (or slow freezing) typically includes gradually cooling a liquid formulation to a temperature suitable for storage at a predetermined rate. Typically, a temperature suitable for storage includes, but is not limited to, a temperature at or lower than about −10° C., −20° C., −30° C., −40° C., −50° C. The gradual step down cooling can be at a rate of approximately 0.6, 0.5, 0.4, 0.3, 0.2, or 0.1° C./minute.

Similarly, controlled thawing (slow thawing) typically includes gradually warming a liquid formulation from a frozen state to a desired temperature at a predetermined rate. Typically, a desired temperature for thawing purposes includes, but is not limited to, a temperature at or higher than about 0° C., 10° C., 20° C., or 30° C. The gradual step warming can be at a rate of approximately 0.6, 0.5, 0.4, 0.3, 0.2, or 0.1° C./minute.

Controlled freeze and thaw may be performed in a container, such as a tube, a bag, a bottle, or any other suitable containers. The containers may be disposable. Controlled freeze and thaw may also be performed in a large scale or small scale. For typical large scale production, a liquid formulation may be frozen in batches of about 1 L through 300 L, for example, 3 L. For typical small scale system, a liquid formulation may be frozen in batches of about 1 ml to 500 ml, for example, 30 ml.

However, in mannitol-containing liquid formulations, the slow freezing and/or thawing allows crystallization of mannitol, which in turn, induces protein aggregation. As used herein, “protein aggregation” is meant formation of high molecular weight (HMW) species including both insoluble species detectable by turbidity measurement and soluble species detectable by size-exclusion chromatography HPLC (SEC-HPLC), cation exchange-HPLC (CEX-HPLC), X-ray diffraction (XRD), modulated differential scanning calorimetry (mDSC) and other means known to one of skill in the art.

It is observed that there is a substantial increase in the percentage of HMW species in mannitol-containing formulations upon multiple freeze and thaw cycles (see the Examples section). Increased amount of mannitol in the formulation also results in higher percentage of HMW species formation. Reduced processing volume appears to maintain the percentage of HMW species formed compared to large scale (e.g., 125 L).

An exothermal event is observed during cooling in mannitol-containing formulations. The observed enthalpy, which is due to the crystallization of mannitol as well as to the unfrozen water, increases as the processing scale increases (freeze and thaw rates decreases), or the mannitol level in the formulation increases. Crystallization event upon thawing in the mannitol-containing formulation is also observed. Without wishing to be bound by theory, the crystallization events in frozen solution suggest that the phase transition due to crystallization may induce the aggregation of protein upon freeze and thaw. Crystallization of mannitol increases with the mannitol level, which corresponds to higher % HMW formation. There was more mannitol crystallization observed in larger process scale simulation than that of the smaller scale, again correlated to greater rate of HMW formation. Decreasing mannitol in the formulation generally favors reducing HMW species formation in the liquid formulation during freeze and thaw.

### Increased Protein Concentration Suppresses Protein Aggregation

The present invention discovered that increasing protein concentration in the liquid formulation suppresses or inhibits protein aggregation during slow freezing and/or thawing process. As described in the Examples section, it was found that increasing protein concentration above 20-30 mg/ml resulted in a decrease in the amount of HMW species formation. Without wishing to be bound by theory, it is contemplated that the increased protein aggregation at low protein concentration (e.g., <20 mg/ml) may be caused by the increased probability of two molecules coming together during freezing and/or thaw. Typically, a protein concentration greater than 50 mg/ml is used to suppress protein aggregation. Preferably, a protein concentration greater than about 75 mg/ml, 100 mg/ml, 125 mg/ml, or 150 mg/ml is used to suppress protein aggregation. More preferably, a protein concentration between 50 mg/ml to 200 mg/ml is used. As used herein, the term “suppresses protein aggregation,” or grammatical equivalents, denotes a reduction of the percentage of HMW species in a liquid formulation as compared to the percentage of HMW species formed in a similar liquid formulation but containing a protein concentration less than 20 mg/ml. The term “suppresses protein aggregation” also includes inhibiting or eliminating formation of HMW species.

Thus, by increasing the protein concentration in the liquid formulation containing mannitol, the present invention allows slow freezing and/or thawing of the liquid formulation without inducing significant protein aggregation. The present invention is particularly useful for storing drug product containing drug substance. For example, the present invention allows all the excipients including mannitol in a drug product to be present during slow freezing and/or thawing process while keeping the drug substance stable and biologically active. Therefore, the present invention eliminates the need for removing mannitol from a drug formulation before storage and adding it back during the drug product filling operation.

Thus, the liquid formulations containing mannitol and a protein concentration higher than 50 mg/ml may be stored directly in that form for later use, stored in a frozen state as an intermediate step and thawed prior to use, or subsequently prepared in a dried form, such as a lyophilized, air-dried, or spray-dried form, for later reconstitution into a liquid form or other form prior to use. In addition, compositions containing biologically active amount of the protein can be prepared and stored directly in their liquid form in accordance with the present application to take full advantage of the convenience, ease of administration without reconstitution, and ability to supply the formulation in prefilled, ready-to-use syringes or as multidose preparations if the formulation is compatible with bacteriostatic agents. The present application also provides other forms of compositions containing biologically active amount of the protein in the liquid formulation stored and prepared as described above.

It should be understood that the above-described embodiments and the following examples are given by way of illustration, not limitation. The liquid formulation of the present invention is applicable to proteins in general. For example, the antibodies used in the liquid formulations described in the Examples section can be any antibodies. Various changes and modifications within the scope of the present invention will become apparent to those skilled in the art from the present description.

### EXAMPLES

### Example 1

### Slow Freeze and Thaw of a Monoclonal Antibody Formulation

A formulation containing a monoclonal antibody (Mab) at various concentrations and 10 mM histidine, 10 mM methionine, 0-4% mannitol and 0-0.02% polysorbate-80, was frozen and thawed multiple times using a CryoPilot (CP) system (Stedim Biosystems). Each freeze and thaw profile included step-down cooling to −55° C., and warming to 32° C. while the solution was mixed.

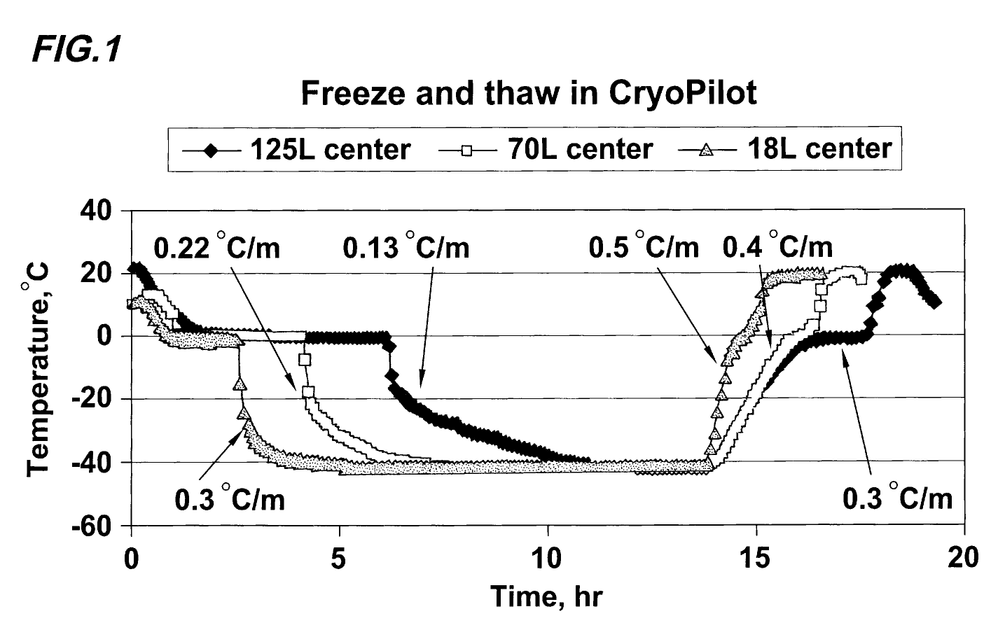

The CP simulates operation of a CryoVessel (Stedim Biosystems), the full scale production unit. The CP set point profiles for various process volumes had been developed prior to this work, to mimic behavior of the CryoVessel. FIG. 1 illustrates a sample of product temperature trace at each process scale with the CP system. Freezing (or thawing) rate was defined as the thermocouple reaching −42° C. from 0° C. (or 0° C. from −42° C.) divided by the time.

Thawed samples were analyzed primarily by SEC-HPLC and CEX-HPLC to evaluate the level of high molecular weight species (% HMW), and track the levels of acidic and basic species. Modulated differential scanning calorimetry (mDSC) and X-Ray Diffraction (XRD) were also used to assess crystallinity and polymorphs of mannitol in frozen solutions.

### Example 2

### Mannitol Induces Protein Aggregation During Slow Freeze and Thaw

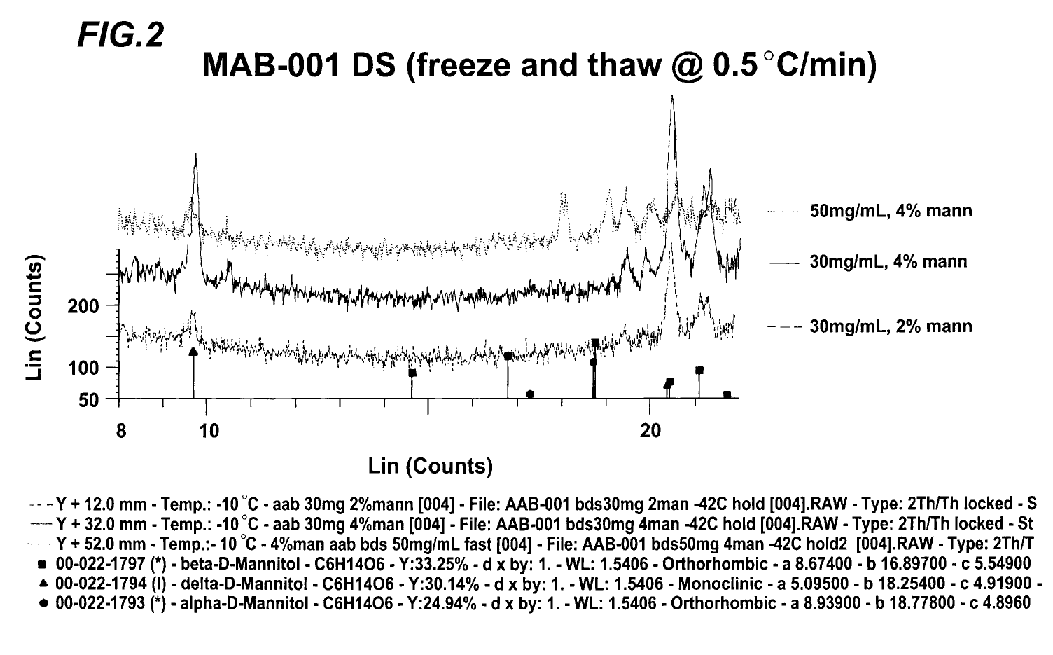

A humanized monoclonal antibody (referred to as MAB-001 in this experiment) was found to aggregate in the presence of mannitol during a slow freeze-thaw process similar to the one described in Example 1. FIG. 2 shows XRD patterns of frozen MAB-001 solutions when cooled to −40° C. then warmed to 20° C. both at 0.5° C./minute. The frozen solution was scanned at −42° C., −30° C. and −10° C. As shown in FIG. 2, the amount of crystallization increased with the amount of mannitol in the formulation and higher protein concentration suppressed mannitol crystallization.

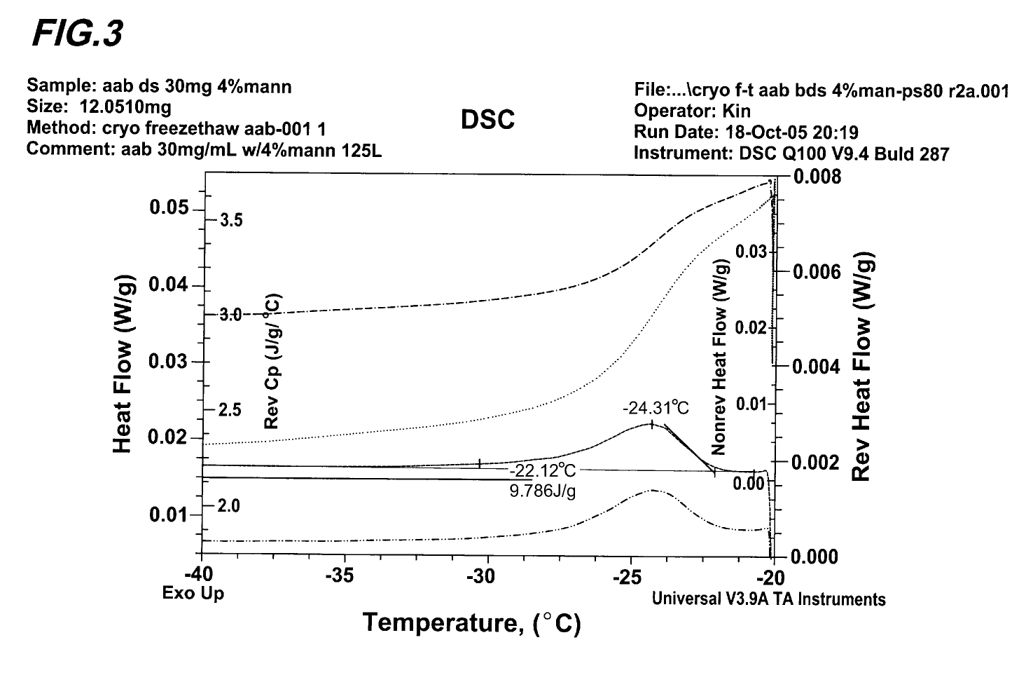

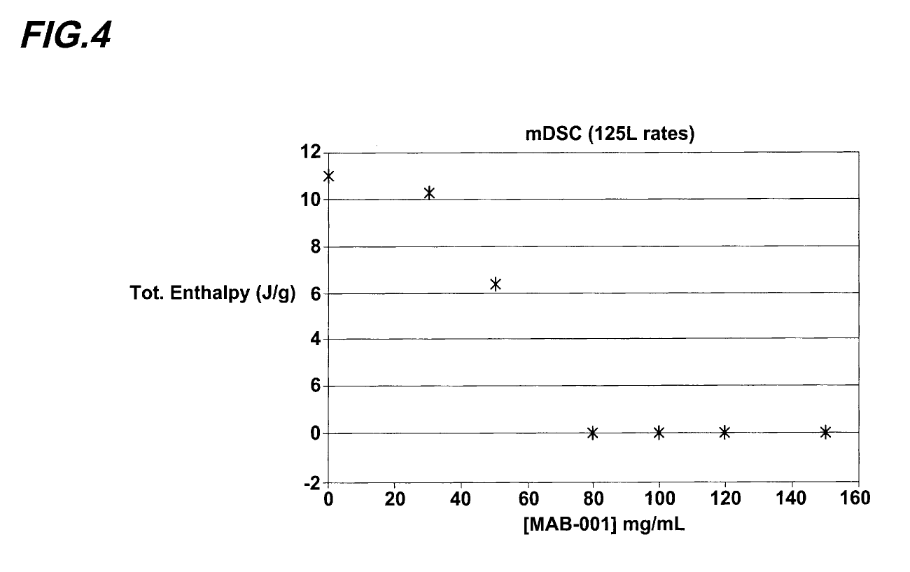

In addition to XRD, modulated Differential Scanning Calorimetry (mDSC) was also used to examine mannitol crystallization in MAB-001 samples. FIG. 3 shows a sample thermogram of mDSC when cooling MAB-001 at concentration of 30 mg/ml down to −42° C. The observed enthalpy (brown trace in FIG. 3) is due to the crystallization of mannitol as shown in FIG. 2. If one assumes total enthalpy equals cooling enthalpy plus warming enthalpy, then the total enthalpy can be plotted against protein concentration as shown in FIG. 4. As also shown in FIG. 4, increased protein concentration (e.g., >30 mg/ml) suppressed the mannitol crystallization.

### Example 3

### Higher Protein Concentration Suppresses Mannitol Crystallization

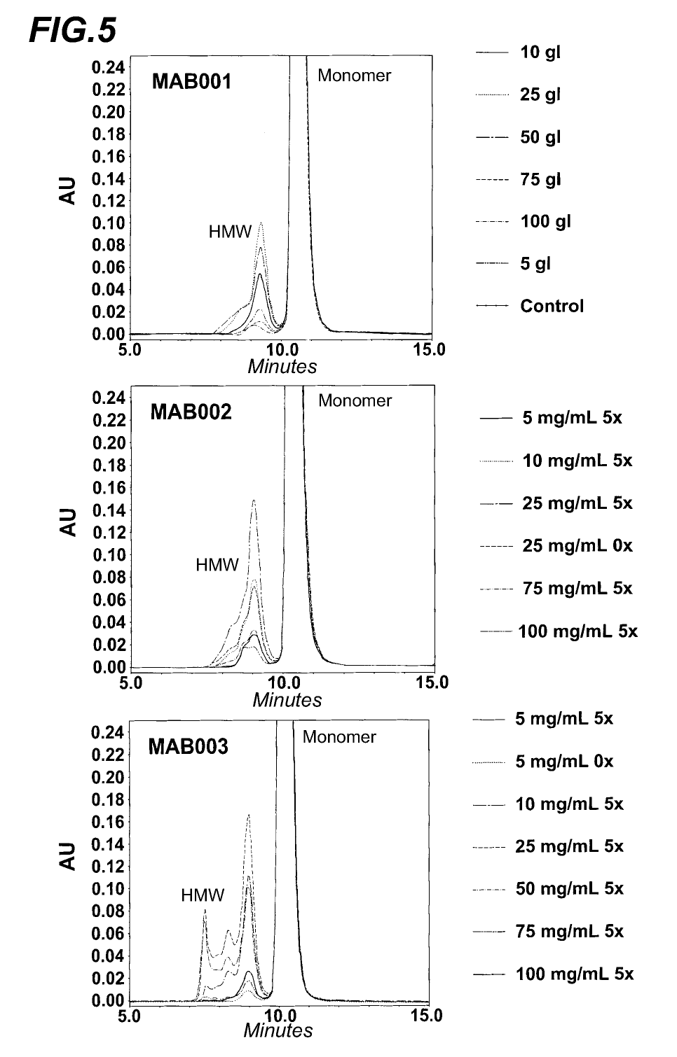

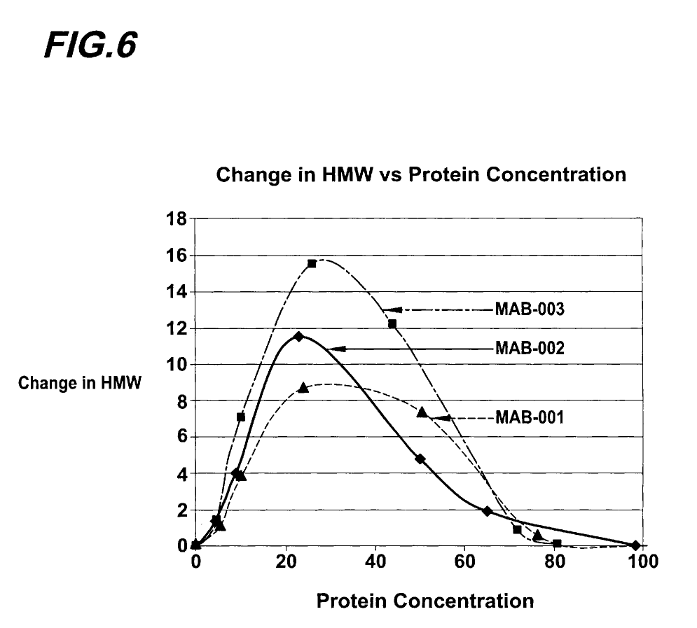

Three antibodies referred to as MAB-001, MAB-002 and MAB-003 were dialyzed into 10 mM histidine, 250 mM mannitol, pH 6.0, then subject to five cycles of freeze-thaw, and monitored for HMW species formation. The SEC-HPLC chromatograms are shown in FIG. 5. As shown in FIG. 5, for each of the three proteins MAB-001, MAB-002 and MAB-003, increased concentration of the protein in the formulation suppressed formation of HMW species. Change in the percentage of HMW species was plotted against the protein concentration in FIG. 6. As shown in FIG. 6, for each of the three proteins, increasing the protein concentration above 20-30 mg/ml decreased the amount of HMW formation detected by SEC-HPLC. This result was consistent with the MDSC data.

### Example 4

### Lab Scale Vs. Production Scale

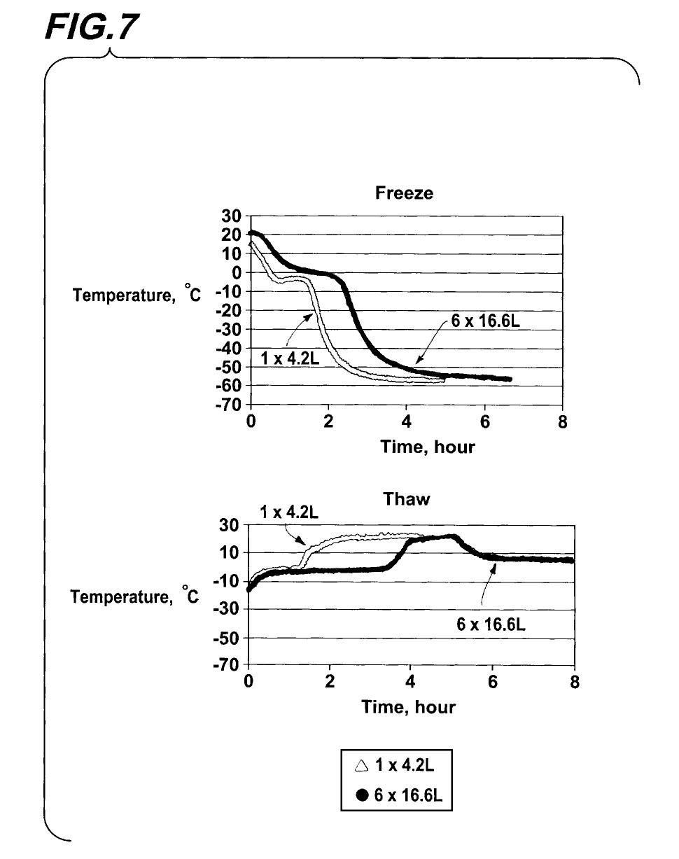

Fast and slow freeze/thaw cycles were developed in a lab system S³ Celsius (Stedim Biosystems) to match product trace of production scale and to bracket the minimum and maximum production loads. FT-100 Celsius (Stedim Biosystems) was used at different production loads. For minimum production load, 4.2 L (1 bag) were used. For maximum production load, 100 L (6 bags at 16.6 L each) were used. Same freeze/thaw set point profile with same mixing speed from FT-100 resulted in 2 sets of product temperature traces based on production loads as shown in FIG. 7.

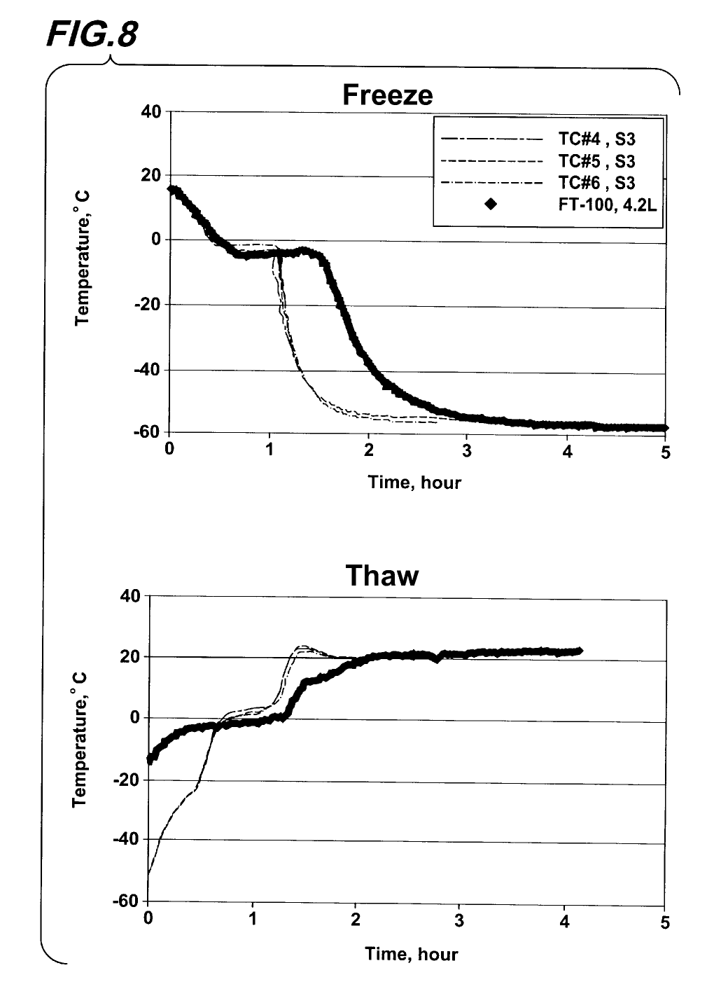

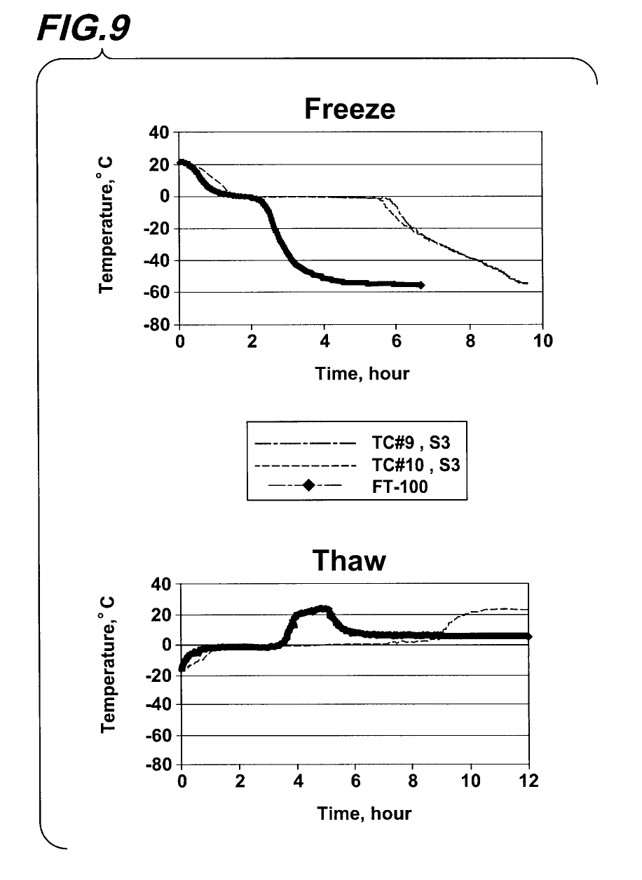

Freeze and thaw cycle development was performed using the lab scale S³ system. As shown in FIG. 8, the rates for faster freeze and thaw of the lab system were faster than the rates at the minimum production load. As shown in FIG. 9, the rates for slow freeze and thaw of the lab system were slower than the rates at maximum production scale.

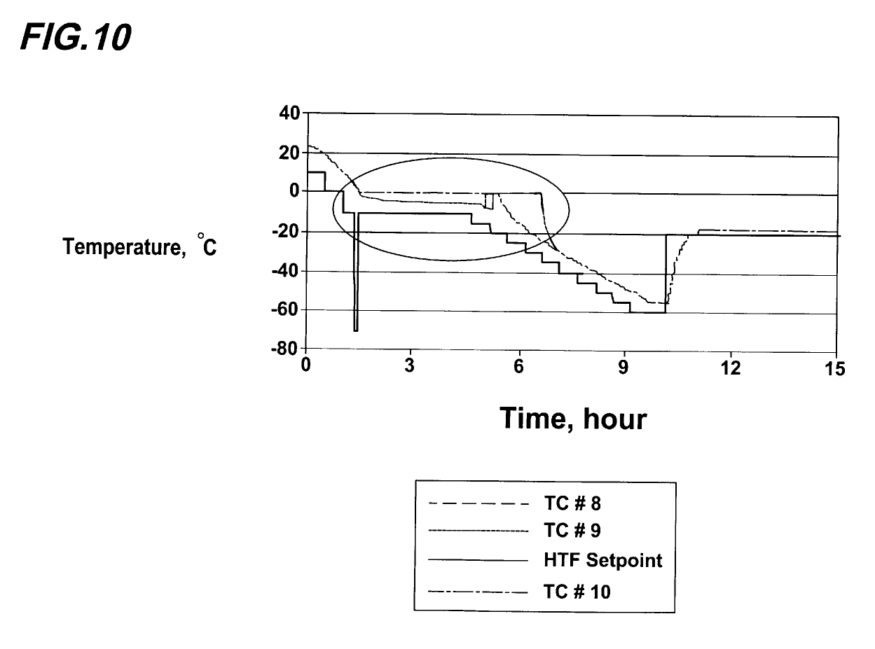

Supercooling was observed during lab scale cycle development for slow freeze and thaw. A typical supercooling phenomenon is shown in FIG. 10. The freeze temperature is depressed when supercooling happens. Super cooling phenomenon was observed in 2 out of 3 trials of slow freeze/thaw cycles (67% occurrence). Supercooling might be a random occurrence with protein and buffer runs or may be caused by bag positions, which were unpredictable. Supercooling affected normal freeze time (NFT) (from 5 C to −5 C) calculation. Supercooling may be related to protein concentration level.

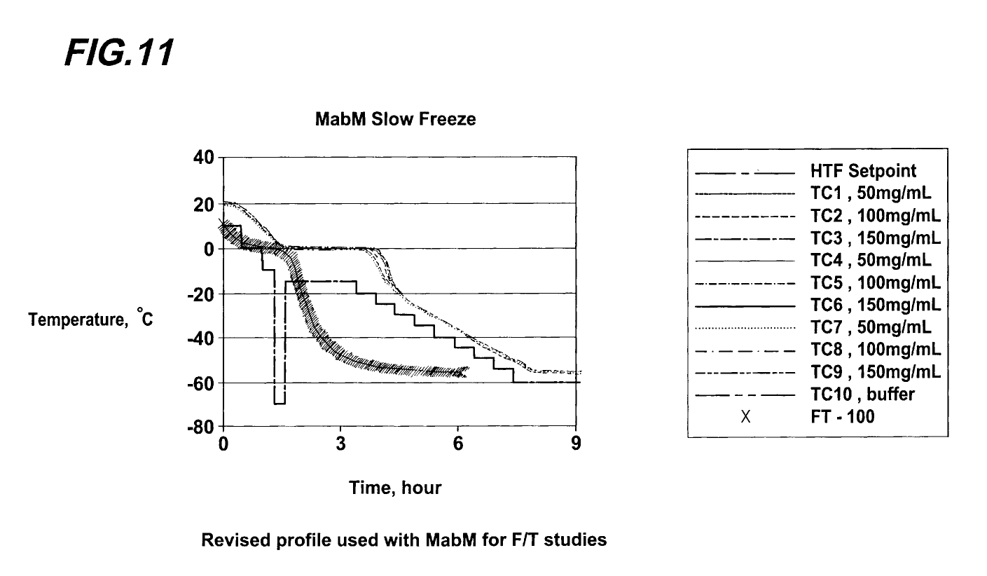

To avoid supercooling, a monoclonal antibody was used as a model protein (MabM) to develop the lab system slow freeze and thaw cycle. MabM was concentrated and dialyzed to Mab formulation buffer solution, then diluted at the following concentrations 50 mg/ml, 100 mg/ml and 150 mg/ml. The formulations without mannitol were prepared, followed by frozen and thawed 5 times using the lab system. Solid mannitol was then added to the mannitol free formulations. These solutions were again frozen and thawed 5 times with the lab system Slow freeze/thaw profile was revised by extending initial deep frozen time and lowering initial freezing temperature to facilitate nucleation. As shown in FIG. 11, the revised profile was performed on 5 buffer trials, followed by 5 MabM trials with or without mannitol (15 total) and no supercooling was observed in any of the 10 thermocouple traces (0% occurrence). Manual mix post thaw was required for all protein concentration levels.

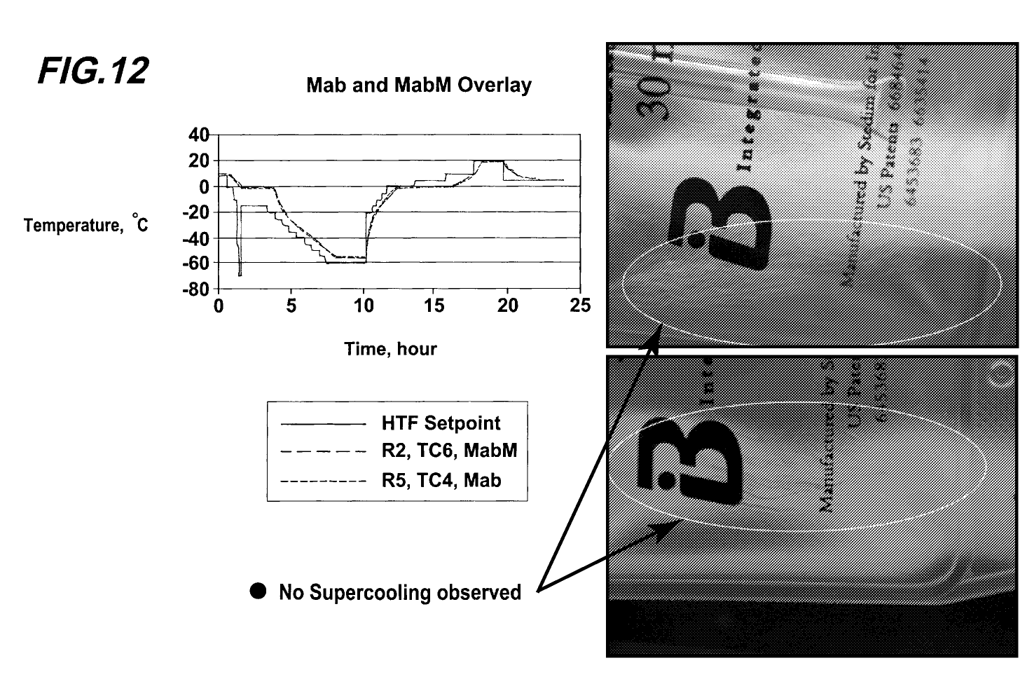

The fast and slow freeze and thaw profiles developed above were used to assess the feasibility of freeze and thaw of monoclonal antibody (Mab) drug substance. The S³ lab scale Celsius was used in this experiment. Assay assessments included visual observation of cryoconcentration, SEC HPLC for HMW and low molecular weight (LMW) species, pH, turbidity by A400, concentration and CEX HPLC. As shown in FIG. 12, the product temperature traces of Mab and MabM overlayed. No supercooling was observed for any of the 10 thermocouple traces up to 5 Mab runs with or without mannitol. Manual mix post thaw was required for all concentration levels.

### Example 5

### Formation of HMW Relates to Freeze and Thaw Rates

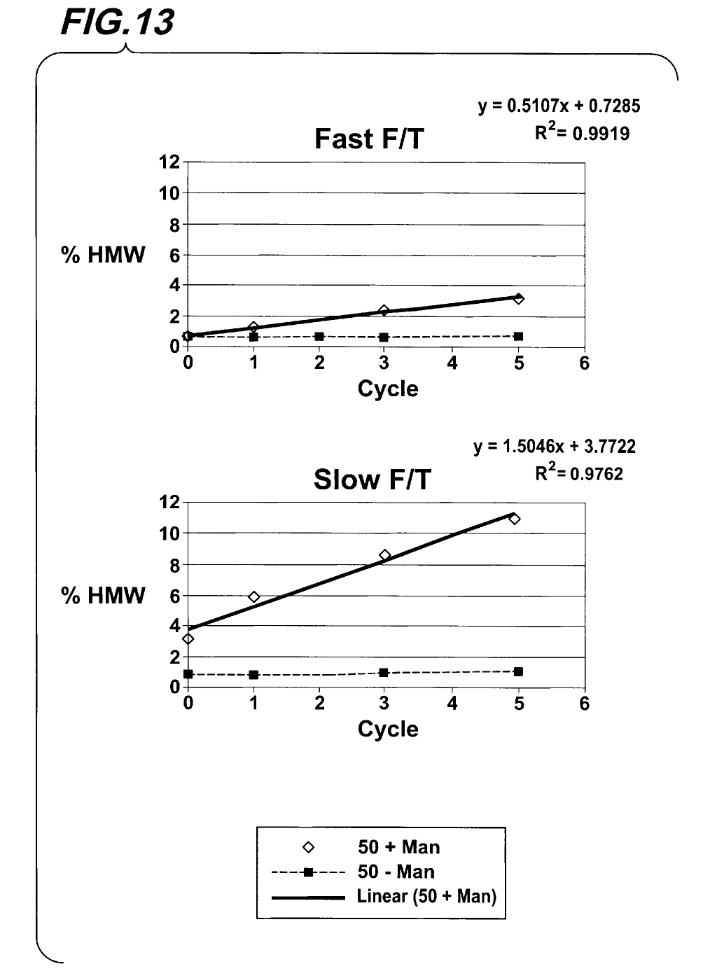

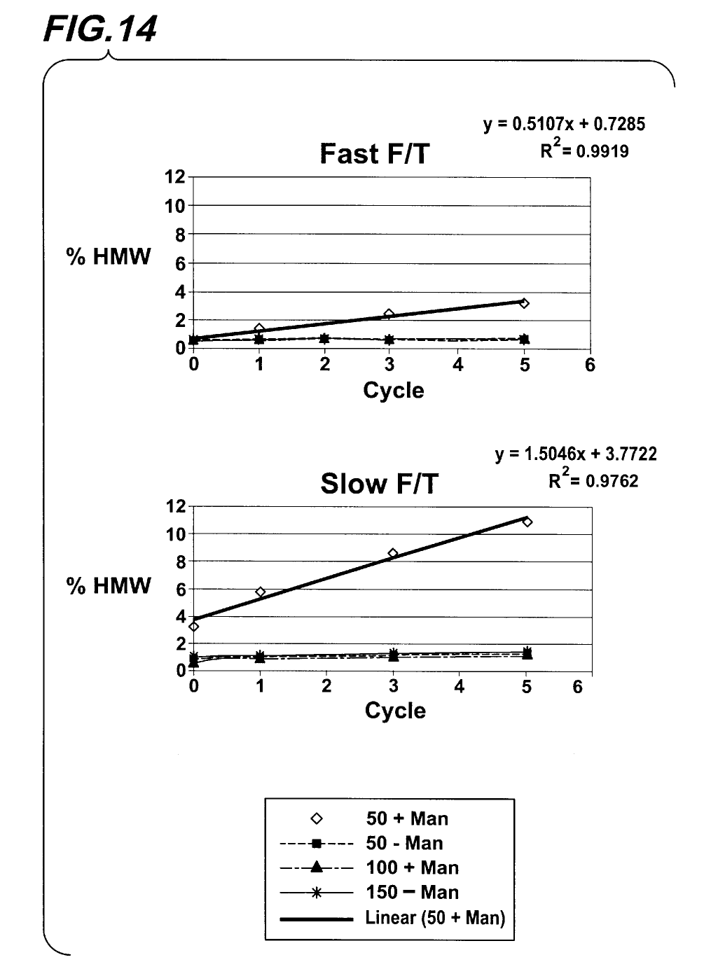

As shown in FIG. 13, the percentage of HMW species increased more significantly after multiple freeze and thaw cycles at slow rates as compared to fast rates. No changes in the amount of LMW species, pH, turbidity, concentration as well as acidic and basic species were observed. As also shown in FIG. 13, the increase of HMW species was only seen in a formulation with protein concentration of 50 mg/ml and containing mannitol. As shown in FIG. 14, no increase in HMW species was observed with Mab formulation at concentration of 100 mg/mL containing mannitol. In addition, it was observed that Mab without mannitol remained stable for up to 5 freeze and thaw cycles in Stedim bags with concentrations of 50 and 150 mg/mL.

### INCORPORATION BY REFERENCE

All publications and patent documents cited in this application are incorporated by reference in their entirety for all purposes to the same extent as if the contents of each individual publication or patent document were incorporated herein.

What is claimed is:

1. A method for storing a liquid formulation, the method comprising gradually cooling the liquid formulation to a temperature lower than −10° C., wherein the liquid formulation comprises mannitol and a protein, the protein being in a concentration greater than 50 mg/ml such that the greater concentration suppresses protein aggregation during cooling.

2. The method of claim 1, wherein the temperature is at approximately −20° C., −40° C., or −50° C.

3. The method of claim 1, wherein the mannitol is in an amount of approximately 0-15%.

4. The method of claim 1, wherein the cooling is at a rate of approximately 0.5° C./minute, 0.3° C./minute, or 0.1° C./minute.

5. The method of claim 1, wherein the protein is in a concentration between 50 mg/ml and 200 mg/ml.

6. The method of claim 1, wherein the protein is in a concentration greater than 75 mg/ml, 100 mg/ml, 125 mg/ml, or 150 mg/ml.

7. The method of claim 1, wherein the protein is an antibody.

8. The method of claim 7, wherein the antibody is a monoclonal antibody.

9. The method of claim 1, wherein the protein is a pharmaceutical drug substance.

10. The method of claim 1, wherein the method is a process intermediate.

11. A method for preparing a liquid formulation, the method comprising gradually warming the liquid formulation from a frozen state to a temperature higher than 0° C., wherein the liquid formulation comprises mannitol and a protein, the protein being in a concentration greater than 50 mg/ml such that the greater concentration suppresses protein aggregation during warming.

12. The method of claim 11, wherein the temperature is at approximately 20° C., or 30° C.

13. The method of claim 11, wherein the mannitol is in an amount of approximately 0-15%.

14. The method of claim 11, wherein the warming is at a rate of approximately 0.5° C./minute, 0.3° C./minute, or 0.1° C./minute.

15. The method of claim 11, wherein the protein is in a concentration between 50 mg/ml and 200 mg/ml.

16. The method of claim 11, wherein the protein is in a concentration greater than 75 mg/ml, 100 mg/ml, 125 mg/ml, or 150 mg/ml.

17. The method of claim 11, wherein the protein is an antibody.

18. The method of claim 17, wherein the antibody is a monoclonal antibody.

19. The method of claim 11, wherein the protein is a pharmaceutical drug substance.

20. The method of claim 11, wherein the method is a process intermediate.

21. A composition comprising a biologically effective amount of the protein in the liquid formulation prepared by the method of claim 11.

22. A method for inhibiting mannitol-induced aggregation of a protein in a liquid formulation, the method comprising increasing the protein concentration to an amount greater than 50 mg/ml.

23. The method of claim 22, wherein the protein is in a concentration greater than 75 mg/ml, 100 mg/ml, 125 mg/ml, or 150 mg/ml.
